(19) United States
(12) Patent Application Publication
(10) Pub. No.: US 2023/0355138 A1
(43) Pub. Date: Nov. 9, 2023

(54) ELECTRONIC DEVICE AND SYSTEM FOR ASSISTING USER MOTION

(71) Applicant: Samsung Electronics Co., Ltd., Suwon-si (KR)

(72) Inventors: Seonghoon KANG, Suwon-si (KR); Hongsik PARK, Suwon-si (KR); Soomi LEE, Suwon-si (KR); Minkyu JUNG, Suwon-si (KR); Sooyoung CHOI, Suwon-si (KR)

(21) Appl. No.: 18/353,365

(22) Filed: Jul. 17, 2023

Related U.S. Application Data
(63) Continuation of application No. PCT/KR2022/001644, filed on Jan. 28, 2022.

(30) Foreign Application Priority Data
Mar. 9, 2021 (KR) 10-2021-0030993

(51) Int. Cl. A61B 5/11; G06V 40/20; A61B 5/00

(52) U.S. Cl. CPC A61B 5/1122; G06V 40/20; A61B 5/742

(57) ABSTRACT

An electronic device may include a communication module and a processor operatively connected to the communication module, wherein the communication module may communicate with a server and an external electronic device including a sensor for measuring user motion, and the processor may control that sensing data obtained by measuring the user motion is acquired from the external electronic device, through the communication module, a motion data set including standard/reference motion data related to another user having a standard/reference body is generated by converting, on the basis of information about the user, motion data including a time-dependent body motion trajectory based on the sensing data, and the motion data set is transmitted to the server through the communication module.

### CROSS-REFERENCE TO RELATED APPLICATIONS

This application is a continuation of International Application No. PCT/KR2022/001644 filed on Jan. 28, 2022, designating the United States, in the Korean Intellectual Property Receiving Office, and claiming priority to KR Patent Application No. 10-2021-0030993 filed on Mar. 9, 2021, in the Korean Intellectual Property Office, the disclosures of all of which are hereby incorporated by reference herein in their entireties.

### BACKGROUND

### Field

Various example embodiments relate to an electronic device and system for assisting a user's motion.

### Description of Related Art

Wearable robots that users may wear on bodies thereof are being developed in various forms according to the purpose of the robot, such as a form that may be worn on a part of the body, a form that may be worn all over the body, and a form that replaces a part of the body. In particular, wearable robots are often developed for the purpose of assisting elderly users to perform specific motions. For example, the wearable robot may be developed for the purpose of rehabilitating a specific body or replacing a specific body in the case that a specific body cannot function.

The wearable robot may assist a body in motion by applying a force to a body in the same direction as a motion direction of the body of a user wearing the same using an actuator or may train the body by applying force to the body in a direction opposite to a direction in which the user's body moves. In such a wearable robot, motions of an actuator are pre-programmed and stored, and the actuator may be driven according to the stored motions.

As we enter an aging society, the wearable robot industry is growing every year, and the demand for wearable robots capable of providing various motions is expected to increase in the future.

### SUMMARY

In a wearable robot, in order to assist a user's motion, an actuator operation may be programmed in various ways.

According to various example embodiments, a second wearable robot worn by a second user may copy motion contents of a first user wearing a first wearable robot to assist a motion of the second user.

For example, an electronic device according to various example embodiments may sense a user's motion to generate a motion data set, and share the motion data set with other users so that other users may perform the same motion. Further, by regenerating the motion data set to suit the user's physical characteristics in consideration of differences in physical characteristics such as physical structure, health condition, and age of users, a personalized motion data set may be provided.

For example, in the case that the user's leg length is longer than the average, a motion trajectory range may be adjusted to be greater than the standard, and in the case that the user's age is higher than the average, a motion speed may be adjusted to be slower than the standard.

It would be a technical issue for manufacturers to provide an electronic device capable of providing various motions and assisting users to perform motions suitable for individual characteristics.

According to various example embodiments, an electronic device may include a communication module comprising communication circuitry; and a processor operatively connected, directly or indirectly, to the communication module, wherein the communication module may be configured to communicate with a server and an external electronic device including a sensor configured to measure a user's motion, wherein the processor may be configured to control to acquire sensing data obtained by measuring the user's motion from the external electronic device through the communication module, to generate a motion data set including standard/reference motion data related to other users having a standard/reference body in a manner of converting motion data including a sequential motion trajectory of the body based on the sensing data based on information of the user, and to transmit the motion data set to the server through the communication module.

According to various example embodiments, an electronic device may include a communication module comprising communication circuitry; a memory configured to store user information; and a processor operatively connected to the communication module and the memory, wherein the communication module may be configured to communicate with a server and an external electronic device including a sensor and an actuator, wherein the processor may be configured to receive a motion data set including standard motion data related to a motion of other users having a standard body from the server through the communication module, to acquire user information stored in the memory, to generate a target motion data set personalized to a user in a manner of converting the motion data set based on the user information, to acquire sensing data obtained by measuring a user's motion from the external electronic device through the communication module, to generate feedback by comparing motion data including a sequential motion trajectory of a body based on the sensing data with the target motion data set, and to transmit a signal for controlling the external electronic device based on the feedback to the external electronic device through the communication module.

According to various example embodiments, a motion assistance system may include a first wearable device including a first sensor; and a second wearable device including a second sensor and an actuator, wherein the first wearable device may be configured to acquire first motion data related to a motion of a first user measured by the first sensor, wherein the second wearable device may be configured to acquire the first motion data, to acquire second motion data related to a motion of a second user measured by the first sensor, and to control the actuator based on a result of comparing the first motion data and the second motion data.

A user can receive data sets of various motions, and be assisted or trained in various motions.

Further, when the user wants to perform a specific motion, the exercise effect can be enhanced by performing the motion in an accurate posture according to a data set generated by a trainer.

Further, the user can perform the same motions as those performed by the trainer in real time and receive feedback in real time to perform accurate motions, thereby increasing the exercise effect.

Further, the user can receive a motion data set corrected to suit the user's body characteristics, thereby receiving motion assistance personalized to the user.

### BRIEF DESCRIPTION OF DRAWINGS

In connection with the description of the drawings, the same or similar reference numerals may be used for the same or similar elements. The above and other aspects, features and advantages of certain embodiments of the present disclosure will be more apparent from the following detailed description, taken in conjunction with the accompanying drawings, in which:

FIG. 1 is a block diagram illustrating an electronic device in a network environment according to various example embodiments.

FIG. 2 is a block diagram illustrating an electronic device according to various example embodiments.

FIG. 3 is a diagram illustrating an external electronic device according to various example embodiments.

FIG. 4A is a flowchart illustrating a method of controlling an electronic device so that a processor generates a motion data set based on sensing data acquired from an external electronic device and transmits the motion data set to a server according to various example embodiments.

FIG. 4B is a diagram illustrating an example of sensing data obtained by a processor from an external electronic device according to various example embodiments.

FIG. 4C is a diagram illustrating an example of motion data generated by a processor and additional information acquired from a user according to various example embodiments.

FIG. 4D is a diagram illustrating an example of generating average motion data and standard motion data based on motion data by a processor according to various example embodiments.

FIG. 4E is a diagram illustrating examples of additional information and standard motion data generated by a processor according to various example embodiments.

FIG. 4F is a diagram illustrating an example of an operation in which a processor transmits a motion data set and a motion-related content to a server according to various example embodiments.

FIG. 5A is a flowchart illustrating a method for a processor to control an external electronic device based on a motion data set and user information according to various example embodiments.

FIG. 5B ([a] and [b]) is a diagram illustrating an example of a screen that displays contents related to a motion by a processor according to various example embodiments.

FIG. 5C is a diagram illustrating an example of an operation of generating a target motion data set based on a motion data set and user information by a processor and transmitting the target motion data set to an external electronic device according to various example embodiments.

FIG. 5D is a diagram illustrating an example of an image including a user and feedback information generated by a processor according to various example embodiments.

FIG. 6A is a flowchart illustrating a method of generating target motion data personalized to a user with a method in which a processor converts a motion data set based on user information according to various example embodiments.

FIG. 6B ([a], [b], and [c]) is a diagram illustrating an example in which a processor generates target motion data personalized to a user in a manner of converting a motion data set based on user information, as illustrated in the flowchart of FIG. 6A according to various example embodiments.

FIG. 7A is a diagram illustrating an example of a constitution of a motion assistance system according to various example embodiments.

FIG. 7B is a flowchart illustrating an operation in which a motion assistance system assists a user's motion according to an example embodiment.

FIG. 7C is a diagram illustrating an example of connection between components of a motion assistance system according to various example embodiments.

FIG. 8A is a diagram illustrating an example in which a first user and a second user perform a motion assistance motion in real time in a motion assistance system according to various example embodiments.

FIGS. 8B, 8C, and 8D are flowcharts illustrating a method of assisting a user's motion in real time by a motion assistance system according to various example embodiments.

### DETAILED DESCRIPTION

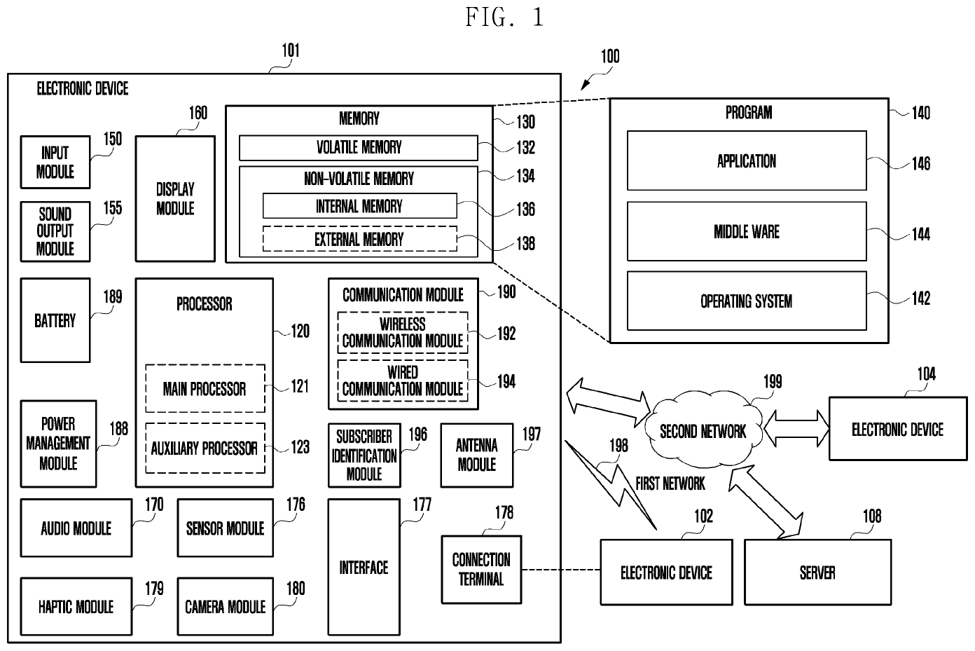

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connection terminal 178, a haptic module 179, a camera module 180, a power management module 188, a battery 189, a communication module 190, a subscriber identification module (SIM) 196, or an antenna module 197. In sore embodiments, at least one of the components (e.g., the connection terminal 178) may be omitted from the electronic device 101, or one or more other components may be added in the electronic device 101. In some embodiments, some of the component (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 1:21 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134. The non-volatile memory may include at least one of an internal memory 136 and an external memory 138.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., the electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

The connection terminal 178 may include a connector via which the electronic device 101 may be physically connected with the external electronic device (e.g., the electronic device 102). According to an embodiment, the connection terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (hDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance a high-frequency band, such as, e.g., beamforming, massive multiple-input and multiple-output (massive MEMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 gigabits per second (Gbps) or more) for implementing eMBB, loss coverage (e.g., 164 decibels (dB) or less) for implementing mMTC, or U-plane latency (e.g., 0.5 milliseconds (ms) or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form an mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIN)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices (e.g. electronic devices 102 and 104 or the server 108). For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an Internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., small home, smart city, smart car, or healthcare based on 5G communication technology or IoT-related technology.

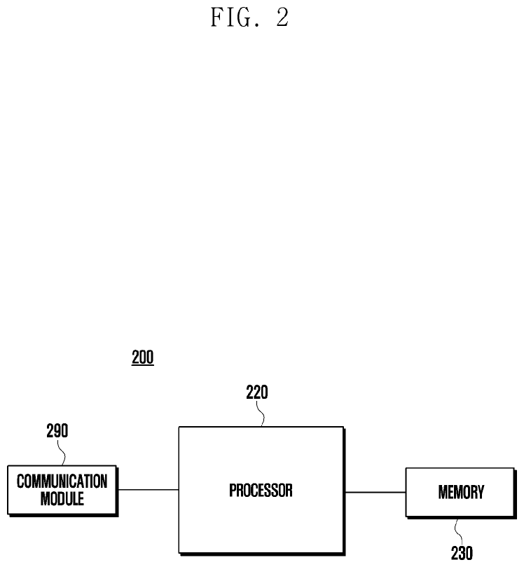

FIG. 2 is a block diagram illustrating an electronic device according to various example embodiments.

With reference to FIG. 2, an electronic device 200 (e.g., the electronic device 101 of FIG. 1) may include a processor 220 (e.g., the processor 120 of FIG. 1), a memory 230 (e.g., the memory 130 of FIG. 1) and/or a communication module 290 (e.g., the communication module 190 of FIG. 1). Components included in FIG. 2 are some of the components included in the electronic device 200, and the electronic device 200 may include various other components, as illustrated in FIG. 1.

The memory 230 may temporarily or non-temporarily store user information. According to an embodiment, the user information may include at least one of body information including a height and body part length, health information including age and health condition, or exercise information including a muscle strength level and flexibility.

The communication module 290 may communicate with an external electronic device and/or a server through a network (e.g., the first network 198 and/or the second network 199 of FIG. 1) to receive and/or transmit various types of information. The processor 220 may be connected, directly or indirectly, to the communication module 290 to process various types of information received by the communication module 290 from the external electronic device and/or the server. Further, the processor 220 may control the communication module 290 to transmit various types of information to the external electronic device and/or the server.

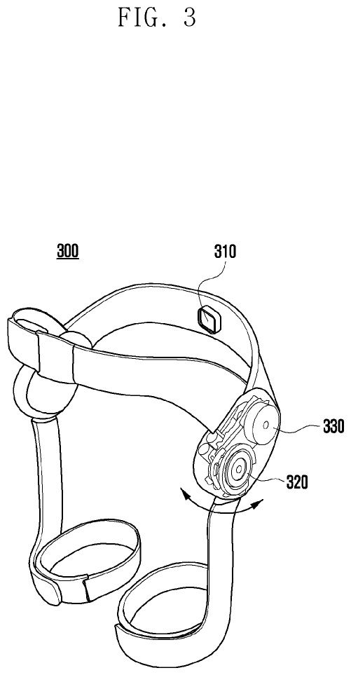

FIG. 3 is a diagram illustrating an external electronic device according to various example embodiments.

According to various embodiments, an external electronic device 300 may include a first sensor 310, a second sensor 320 and/or an actuator 330. Further, the external electronic device 300 may include a physical device assisting the user to wear it on the body (e.g., leg, arm, and waist).

According to various embodiments, the first sensor 310 may include an inertial sensor (IMU). According to an embodiment, the first sensor 310 may be an inertial sensor that measures the degree of inclination of the sensor using a combination of an accelerometer, a rotational speed meter, and/or a magnetometer. For example, the first sensor 310 may measure an inclination angle of a part of the user's body from a reference point.

According to various embodiments, the second sensor 320 may include an encoder sensor. According to an embodiment, the second sensor 320 may include a rotary encoder sensor that detects a rotational motion according to a motion form of an object. For example, the rotary encoder sensor is a sensor that converts a mechanical rotation range into an electrical pulse and may be a sensor that measures a rotation amount and rotation speed of the device. For example, the second sensor 320 may measure a rotation angle of a part of the user's body based on a designated position.

According to various embodiments, the actuator 330 may be a mechanical device that moves or controls the system. For example, by adding mechanical force to a device mounted in a part of the user's body, the actuator 330 may assist in moving the user's body or may apply a load to the user's body.

The external electronic device mentioned in this document is not limited to the form disclosed in FIG. 3 and may include various types of wearable robots capable of being worn by the user and assisting the user's motion.

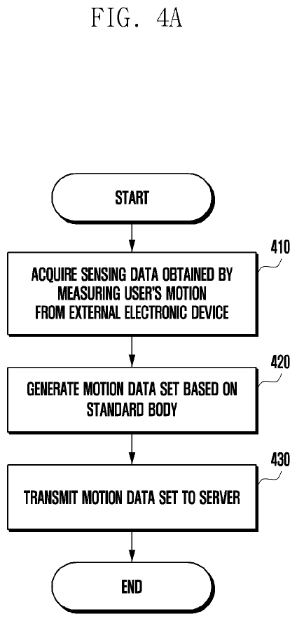

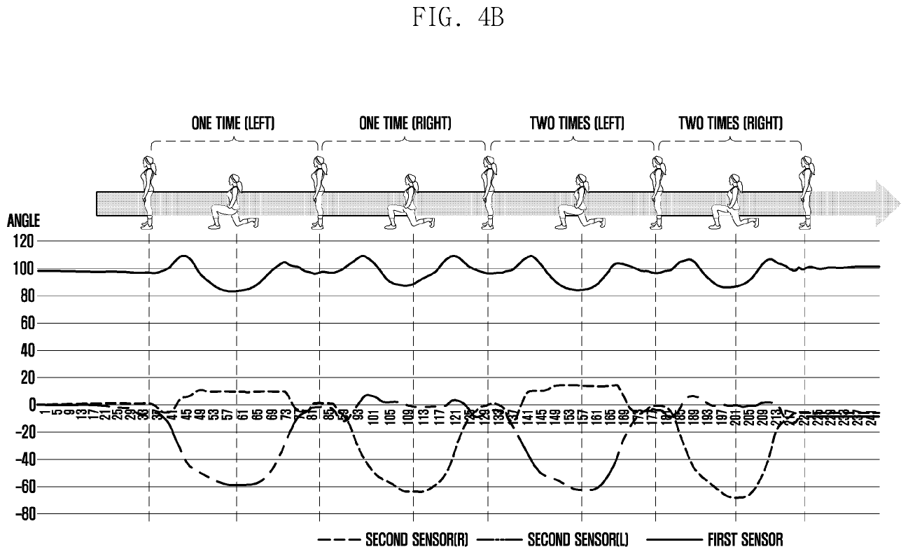

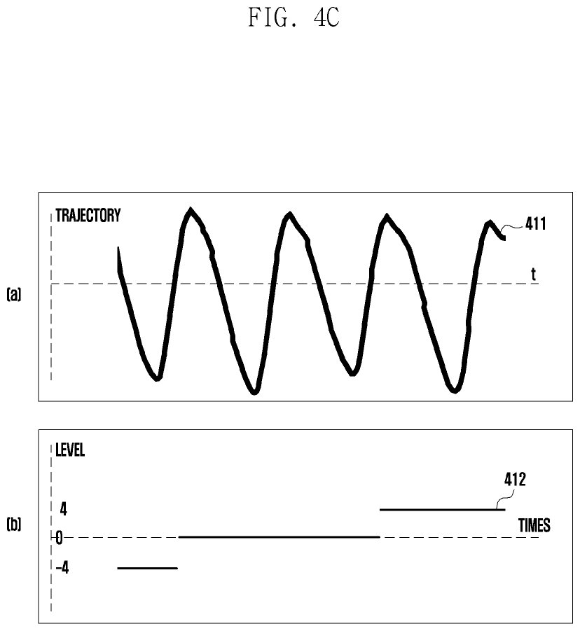

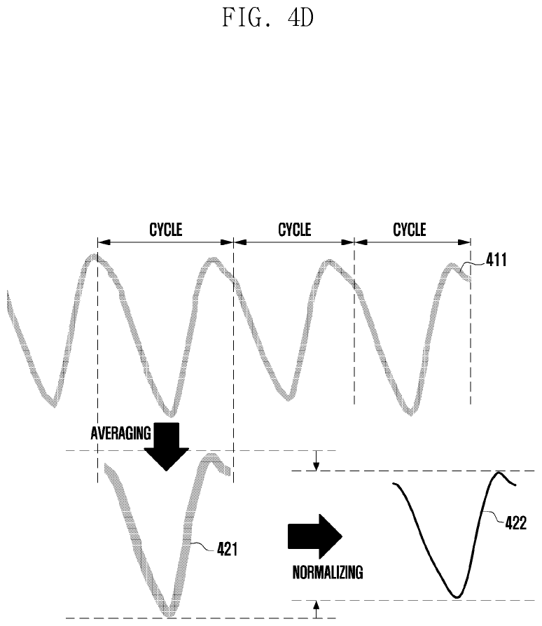

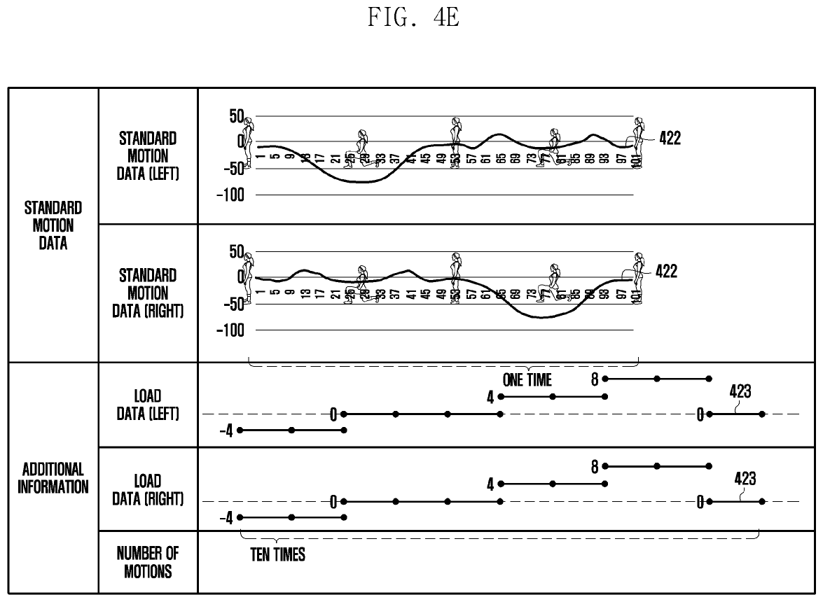

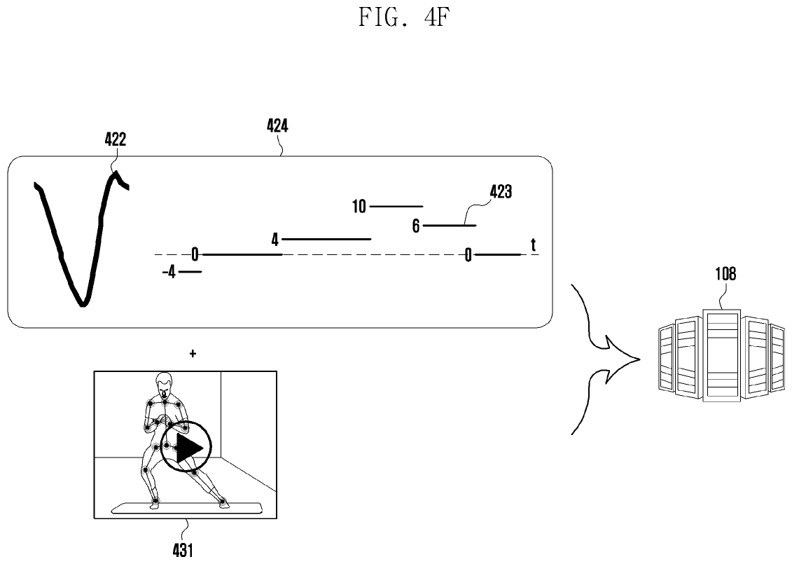

FIG. 4A is a flowchart illustrating a method of controlling an electronic device (e.g., the electronic device 200 of FIG. 2) so that the processor (e.g., the processor 220 of FIG. 2) generates a motion data set based on sensing data acquired from the external electronic device and transmits the motion data set to the server according to various example embodiments.

According to various embodiments, in operation 410, the processor 220 may acquire sensing data obtained by measuring a value related to a user's motion from the external electronic device (e.g., the external electronic device 300 of FIG. 3).

According to an embodiment, the processor 220 may acquire sensing data from the external electronic device 300 through the communication module (e.g., the communication module 290 of FIG. For example, the processor 220 may acquire sensing data received by the communication module 290 from the external electronic device 300 through wireless communication (e.g., short-range communication such as Bluetooth or WiFi).

According to an embodiment, the sensing data may include a value related to a state of the body (e.g., an inclined angle and/or a moved angle of the body) of a user wearing the external electronic device 300 measured by various sensors (e.g., the first sensor 310 and/or the second sensor 320 of FIG. 3) of the external electronic device 300.

According to an embodiment, the processor 220 may generate motion data related to a motion of the body based on the sensing data. For example, the processor 220 may generate motion data (e.g., a sequential motion trajectory of the body) based on a value measured by the first sensor 310 and/or a value measured by the second sensor 320 included in the sensing data.

According to various embodiments, in operation 420, the processor 220 may generate a motion data set based on a standard body.

According to an embodiment, the processor 220 may generate a motion data set including standard motion data related to other users having a standard body and/or additional information related to a level of difficulty of a motion input from the user in a manner of converting motion data based on a designated body.

According to an embodiment, the processor 220 may analyze motion data related to multiple times of motion to distinguish motions constituting one cycle (one time). By dividing motion data into one cycle of a motion and calculating an average of values in one cycle, the processor 220 may generate average motion data. The average motion data may include a value (e.g., motion trajectory range and/or motion execution speed) related to a motion in one cycle.

According to an embodiment, the processor 220 may acquire the user's body information (e.g., body height, body part length, flexibility factor, health condition factor, muscle strength level, and age) stored in the memory 230. According to an embodiment, the processor 220 may generate standard/reference motion data related to other users having a standard/reference body in a manner of converting average motion data based on designated body information. For example, standard/reference body information may be information including various information of a designated body (e.g., height, body part length, flexibility factor, health condition factor, muscle strength level, and age of a standard body based on male and female standard body (e.g., ISO 7250)). Standard/reference body information is body information stored in the memory 230, and the processor 220 may convert standard body information into standard motion data based on a comparison result between the user's body information and the standard body information. For example, the processor 220 may normalize average motion data to generate standard motion data (e.g., motion trajectory range and/or motion execution speed) in a manner of comparing the standard body information with the user's body information and correcting the average motion data corresponding to the difference between body information.

According to an embodiment, the processor 220 may receive an input of additional information related to a motion from the user. For example, the additional information may include a load level of a motion and the number of repetitions of the motion. According to an embodiment, the processor 220 may display a screen for user input on the display (e.g., the display module 160 of FIG. 1) and receive an input of additional information through the screen.

According to various embodiments, in operation 430, the processor 220 may transmit a motion data set to the server.

According to an embodiment, the processor 220 may transmit the motion data set to the server through the communication module 290. For example, the processor 220 may control the communication module 290 to transmit the motion data set to the server through wireless communication (e.g., internet such as WiFi or LTE).

FIG. 4B is a diagram illustrating an example of sensing data obtained by the processor 220 from the external electronic device according to various example embodiments.

With reference to a graph of FIG. 4B, a horizontal axis thereof may represent a time, and a vertical axis thereof may represent an angle of the sensor. The sensing data may include values (e.g., inclined angle and/or moved angle of the body) related to motions of various bodies (e.g., pelvis and legs) of the user wearing an external electronic device measured by various sensors (e.g., the first sensor 310 and/or the second sensor 320 of FIG. 3) of the external electronic device (e.g., the external electronic device 300 of FIG. 3). For example, the user may perform a motion (e.g., lunge motion) of alternately bending left and right legs close to 90 degrees while wearing the external electronic device 300. The first sensor 310 provided in the external electronic device 300 may measure an angle of the pelvis while the user performs a motion, the second sensor (left) may measure a moving angle of the left leg, and the second sensor (right) may measure a moving angle of the right leg. For example, a sensing data graph of FIG. 4C may represent an inclined angle of the user's pelvis and a moved angle of the left and right legs during the lunge motion. With reference to the graph of the value measured by the first sensor, the first sensor value may be measured as about 100 degrees in a state in which the user's leg is extended, and the first sensor value may be measured as about 80 degree in a state in which the user's leg is bent. Further, in a state in which the user's leg is extended, a value of the second sensor (L) and a second sensor (R) may be measured as about 0 degrees, and in a state in which the user's left leg is bent forward and in which the user's right leg is bent downward, a value of the second sensor (L) may be measured as about −60 degrees (rotation of 60 degrees in a forward direction based on 0 degrees), and a value of the second sensor (R) may be measured as about 10 degrees (rotation of 10 degrees in a backward direction based on 0 degrees).

FIG. 4C is a diagram illustrating an example of motion data generated by the processor 220 and additional information acquired from the user according to various example embodiments.

Motion data 411 may include a value related to a motion of the user wearing the external electronic device. With reference to a graph (a), a horizontal axis thereof represents a time (t), a vertical axis thereof represents a motion trajectory, and the motion data 411 may include a sequential motion trajectory value of the body. For example, the motion data 411 may represent a motion trajectory value of the leg in a negative (−) value when an angle between the pelvis and the leg becomes smaller, and a positive (+) value when an angle between the pelvis and the leg becomes larger, or vice versa based on when the pelvis and the leg are aligned based on a value (e.g., an inclined angle of the pelvis) measured by a first sensor and a value (e.g., a moved angle of the leg) measured by a second sensor in sensing data. For example, the motion data 411 may be data (e.g., the degree of leg movement corrected by an inclined angle of the pelvis) related to a motion trajectory obtained by correcting the value (e.g., the moved angle of the leg) measured by the second sensor based on the value (e.g., the inclined angle of the pelvis) measured by the first sensor.

According to an embodiment, the processor 220 may receive an input of additional information related to a level of difficulty of the motion from the user. Additional information 412 may include a load level of a motion and/or the repetition number of the motion. With reference to the graph (h), a horizontal axis thereof represents the number of motions, a vertical axis thereof represents a load level, and the additional information 412 may include load level information according to the number of motions. For example, the additional information 412 may configure a load level in a manner of configuring a level at which the external electronic device does not apply a load to the user to 0, representing a positive (+) value when the external electronic device 300 applies a load in a direction opposite to that of the user's motion, and representing a negative (−) value when the external electronic device assists the user's motion by applying a load in the same direction as that of the user's motion.

FIG. 4D is a diagram illustrating an example of generating average motion data and standard motion data based on motion data by the processor 220 according to various example embodiments.

According to an embodiment, the processor 220 may analyze motion data 411 related to multiple times of motion to distinguish motions constituting one cycle (one time). The processor 220 may classify the motion data 411 into one cycle of a motion and calculate an average of values in one cycle to generate average motion data 421. The average motion data 421 may include a sequential motion trajectory of the body in one cycle.

According to an embodiment, the processor 220 may acquire the user's body information (e.g., body height, body part length, flexibility factor, health condition factor, muscle strength level, and age) stored in the memory 230.

According to an embodiment, the processor 220 may generate standard motion data 422 related to other users having a standard body in a manner of converting the average motion data 421 based on designated body information. For example, standard body information may be information including various information of a designated body (e.g., height, body part length, flexibility factor, health condition factor, muscle strength level, and age of a standard body based on male and female standard body (e.g., ISO 7250)). The standard body information is body information stored in the memory 230, and the processor 210 may convert the standard body information into standard motion data based on a comparison result between the user's body information and the standard body information. For example, the processor 220 may normalize average motion data to generate the standard motion data 422 in a manner of comparing the standard body information with the user's body information and correcting the average motion data 421 corresponding to the difference between body information.

FIG. 4E is a diagram illustrating examples of additional information and standard motion data generated by the processor 220 according to various example embodiments.

With reference to a standard motion data graph of FIG. 4E, the standard motion data 422 may represent a sequential motion trajectory of left and right legs in one cycle of a motion (e.g., lunge motion) based on the standard body. With reference to the graph of the standard motion data 422 (left/right), in a horizontal axis 0, based on the trajectory being 0 in a state in which the pelvis and legs are aligned, in a horizontal axis 30, the standard motion data 422 may represent a trajectory of aligning (trajectory: about 0) the right leg with the pelvis while the left leg bends (trajectory: about −80) close to the pelvis, and in a horizontal axis 80, the standard motion data 422 may represent a trajectory of aligning (trajectory: about 0) the left leg with the pelvis while the right leg bends (trajectory: about −80) close to the pelvis.

With reference to a load level graph of FIG. 4E, a load level 423 may represent a level at which the external electronic device applies a load on a user with a standard body in one cycle of a lunge motion. With reference to the load level graph (left/right), at the beginning of the motion, the external electronic device 300 may assist the user's motion by applying a load in the same direction (−) as that of the user's motion, and represent a sequence that may train the user's body by gradually applying a load in a direction (+) opposite to that of the user's motion.

Further, the number of standard motions in the embodiment of FIG. 4E is 10 times, which may indicate that the motion of one cycle of a lunge motion is repeated 10 times.

FIG. 4F is a diagram illustrating an example of an operation in which the processor 220 transmits a motion data set and a motion-related content 431 to the server according to various example embodiments.

According to an embodiment, the processor 220 may generate a motion data set 424 including standard motion data 422 and/or additional information including a load level 423 and the number of motions. For example, a motion included in the motion data set may be the same motion as that included in the motion-related content 431.

According to an embodiment, the processor 220 may acquire the motion-related content 431 and transmit the motion-related content 431 together with the motion data set 424 to a server 108 through the communication module 290.

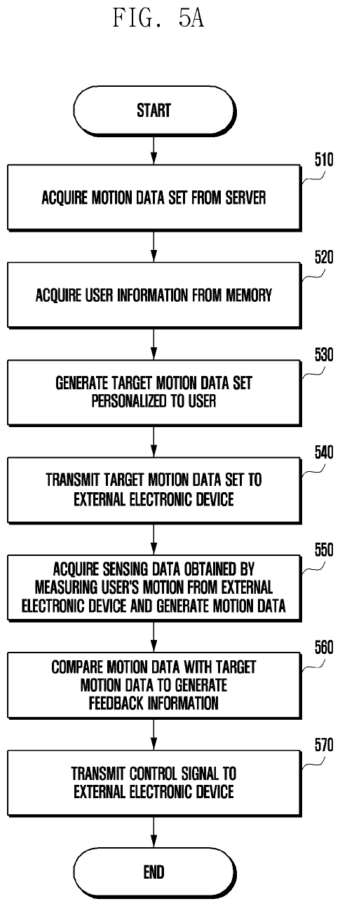

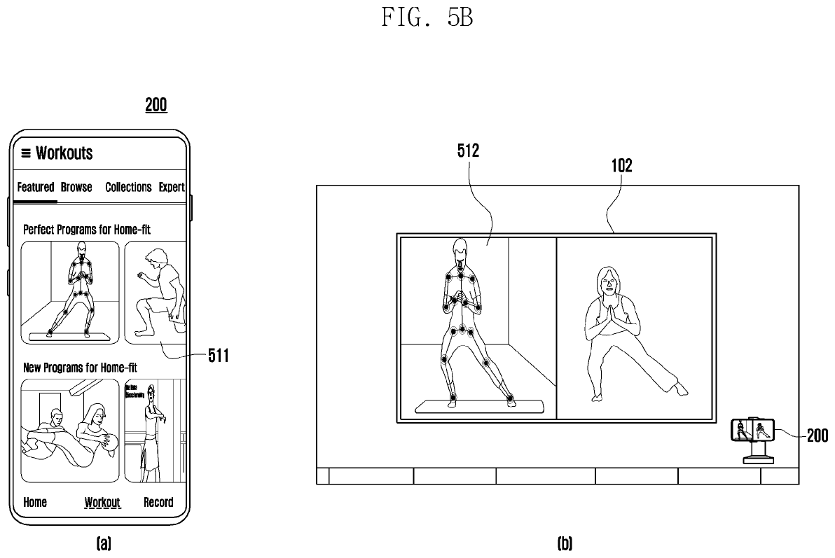

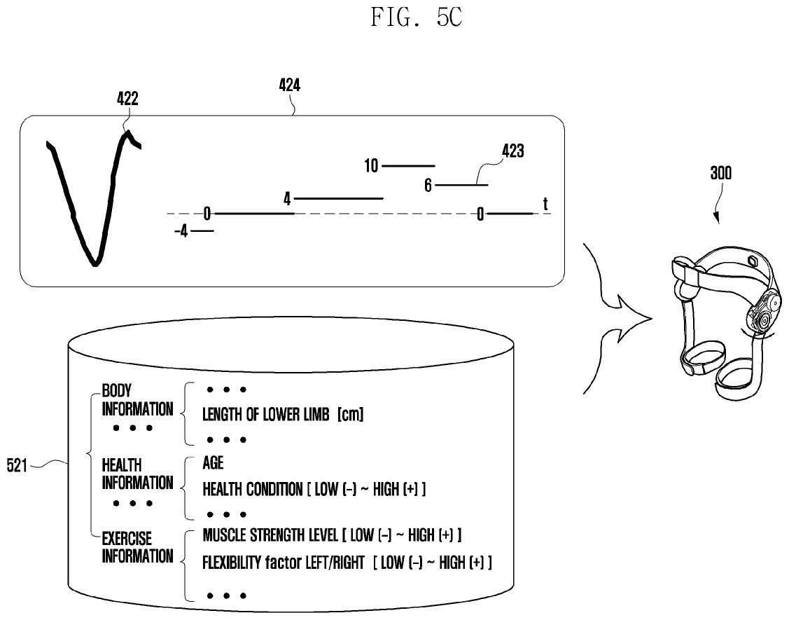

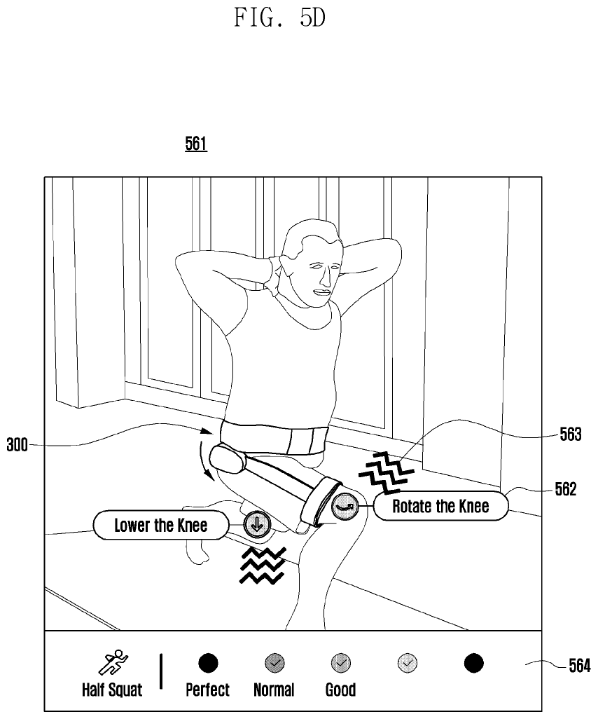

FIG. 5A is a flowchart illustrating a method for the processor (e.g., the processor 220 of FIG. 2) to control the external electronic device (e.g., the external electronic device 300 of FIG. based on a motion data set and user information according to various example embodiments.

According to various embodiments, in operation 510, the processor 220 may acquire a motion data set from the server.

According to an embodiment, the processor 220 may acquire the motion data set from the server through the communication module (e.g., the communication module 290 of FIG. 2). For example, the processor 220 may control the communication module 290 to download a motion data set from the server through wireless communication (e.g., WiFi, LTE).

According to an embodiment, the motion data set may include standard motion data related to other users having a standard body and/or additional information related to a level of difficulty of motions input from the user in a manner of converting motion data related to motions of the body based on a designated body.

According to an embodiment, the processor 220 may acquire a motion-related content from the server through the communication module 290 together with the motion data set. For example, the motion-related content may be an image including a user performing the motion.

According to an embodiment, the motion-related content and the motion data set may be in the form of downloading a file uploaded on the server. For example, the user may download a motion data set or download together a motion-related content and a motion data set while accessing the server (e.g., application, site) on the electronic device 200, selecting a motion-related content to be performed, and streaming the motion-related content.

According to an embodiment, the motion-related content and the motion data set may be in the form of being connected to a specific user on the server and receiving a motion data set and a motion-related content generated by a specific user in real time. For example, the user may access the server (e.g., application, site) on the electronic device 200 to be connected to a specific user and to receive an image including the specific user and a motion data set generated by the specific user using the external electronic device.

According to various embodiments, in operation 520, the processor 220 may acquire user information from the memory (e.g., the memory 230 of FIG. 2).

According to an embodiment, the user information may include at least one of body information including a height and body part length, health information including age and health condition, or exercise information including muscle strength level and flexibility.

According to an embodiment, the user information may be information input by a user or information generated by data accumulated in the memory 230.

According to various embodiments, in operation 530, the processor 220 may generate a target motion data set personalized to the user.

According to an embodiment, the target motion data set may include target motion data personalized to the user in a manner of converting based on user information and/or target additional information personalized to the user in a manner of converting based on user information.

According to an embodiment, the processor 220 may generate target motion data personalized to the user in a manner of converting the motion data set based on user information. For example, the processor 220 may adjust a motion speed based on the length of the user's body part and/or the user's age. For another example, the processor 220 may adjust a load level based on the user's muscle strength level and/or health condition. For another example, the processor 220 may correct a motion trajectory range based on the user's flexibility and/or the user's age.

According to an embodiment, the processor 220 may generate target additional information personalized to the user in a manner of converting additional information based on user information. For example, the processor 220 may adjust the load level based on the user's muscle strength level and/or health condition.

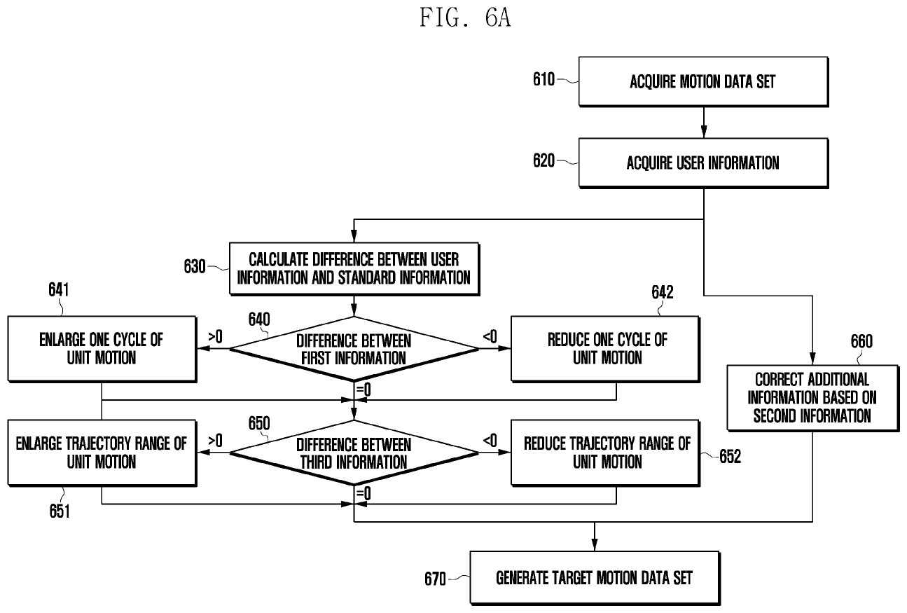

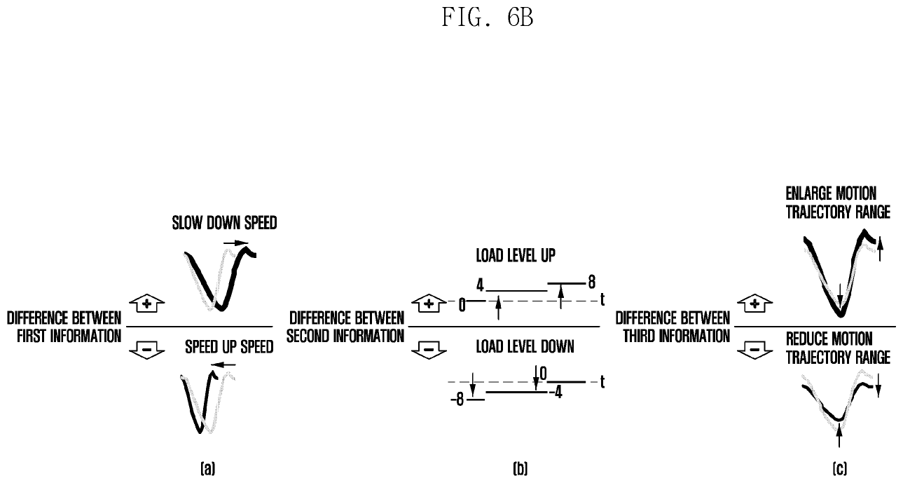

A specific embodiment related to operation 530 may be described in the description related to FIGS. 6A and 6B.

According to various embodiments, in operation 540, the processor 220 may transmit the target motion data set to the external electronic device 300.

According to an embodiment, the processor 220 may transmit the target motion data set to the external electronic device 300 through the communication module 290. For example, the processor 220 may control the communication module 290 to transmit the target motion data set to the external electronic device through wireless communication (e.g., Bluetooth or WiFi).

According to an embodiment, the external electronic device 300 may control the actuator (e.g., the actuator 330 of FIG. 3) based on a target motion data set. For example, the external electronic device 300 may adjust the torque strength and direction of the actuator 330 based on the target motion data set to assist the user to perform the target motion.

According to various embodiments, in operation 550, the processor 220 may acquire sensing data obtained by measuring a user's motion from an external electronic device and generate motion data based on the sensing data.

According to an embodiment, the processor 220 may acquire sensing data from the external electronic device 300 through the communication module 290. For example, the processor 220 may acquire sensing data received by the communication module 290 from the external electronic device 300 through wireless communication (e.g., short-range communication such as Bluetooth or WiFi).

According to an embodiment, the sensing data may include a value related to a state of the body (e.g., an inclined angle and/or a moved angle of the body) of a user wearing the external electronic device 300 measured by various sensors (e.g., the first sensor 310 and/or the second sensor 320 of FIG. 3) of the external electronic device 300.

According to an embodiment, the processor 220 may generate motion data related to a motion of the body based on the sensing data. For example, the processor 220 may generate motion data (e.g., a sequential motion trajectory of the body) based on a value measured by the first sensor 310 and/or a value measured by the second sensor 320 included in the sensing data.

According to various embodiments, in operation 560, the processor 220 may compare motion data with target motion data to generate feedback information.

According to an embodiment, in the case that an absolute value of the difference between the motion data and the target motion data is greater than or equal to a designated value, the processor 220 may determine that the user is performing an erroneous motion. Further, the processor 220 may generate feedback information for guiding the user to perform a target motion corresponding to the determination. For example, the feedback information may be in various forms such as image feedback, haptic feedback, and voice feedback.

According to an embodiment, the processor 220 may display an image including a user on the display (e.g., the display module 160 of FIG. 1) and/or the external electronic device including the display. For example, the processor 220 may acquire an image including a user taken by a camera e.g., the camera module 180 of FIG. 1) to display the image on the display 160 or the external electronic device. Further, the processor 220 may overlay and display image feedback on the image. For example, the processor 220 may overlay and display image feedback in the form such as a figure or a text at a position of a user's body part requiring feedback on an image including the user.

According to an embodiment, the processor 220 may output voice feedback to the external electronic device including an audio module and/or an audio device (e.g., the audio module 170 of FIG. 1).

According to various embodiments, in operation 570, the processor 220 may transmit a control signal to the external electronic device 300.

According to an embodiment, the processor 220 may transmit a signal for controlling the external electronic device 300 to generate a haptic vibration based on feedback information to the external electronic device 300 through the communication module 290. For example, the processor 220 may transmit a control signal to the external electronic device 300 so that a haptic sensor of the external electronic device 300 corresponding to a body part of the user requiring feedback vibrates,

FIG. 5B is a diagram illustrating an example of a screen that displays a content related to a motion by the processor 220 according to various example embodiments.

According to an embodiment, the processor 220 may acquire a motion-related content from the server through the communication module 290 together with the motion data set. For example, the motion-related content may include an image including a user performing a motion. For example, a motion included in the motion data set may be the same motion as that included in the motion-related content.

With reference to part (a) of FIG. 5B, a method of providing a motion-related content 511 and a motion data set may be a form of downloading a file uploaded on the server. For example, the user may download the motion data set or download together the motion-related content 511 and the motion data set while accessing the server (e.g., application, site) on the electronic device 200, selecting a content 511 related to a motion to be performed, and streaming the motion-related content 511. According to an embodiment, the processor 220 may control the external electronic device 102 to display the motion-related content 511 on the display 160 of the electronic device 200 or to display the motion-related content 511 on the external electronic device 102 including the display.

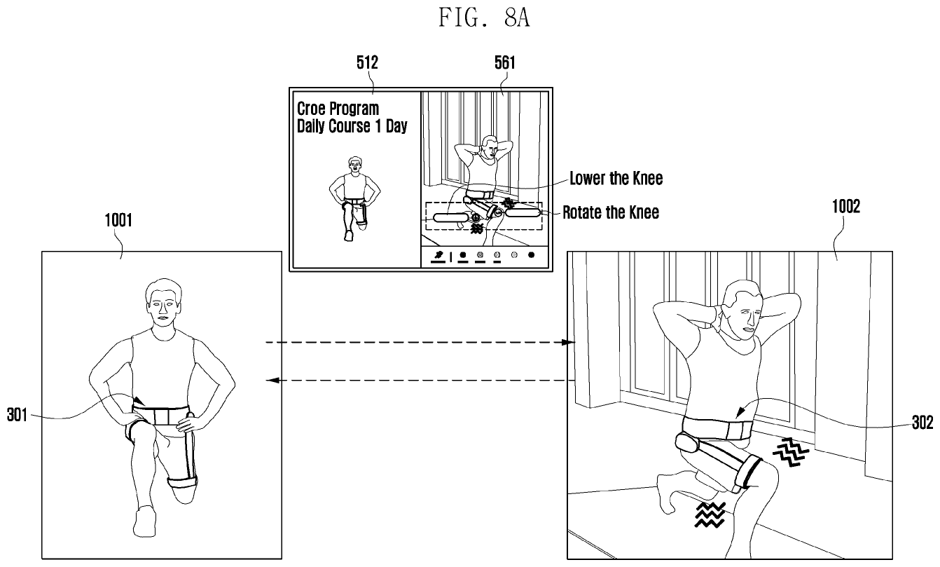

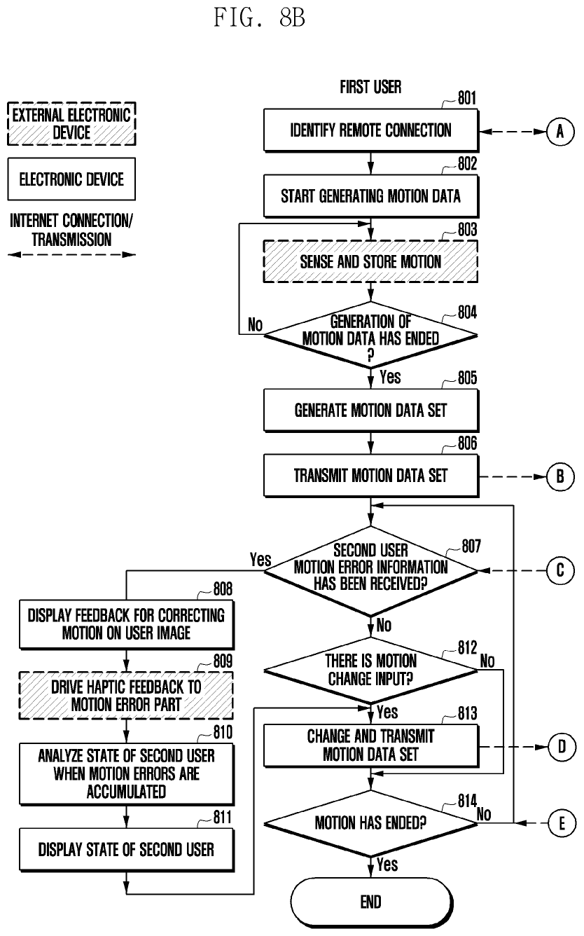

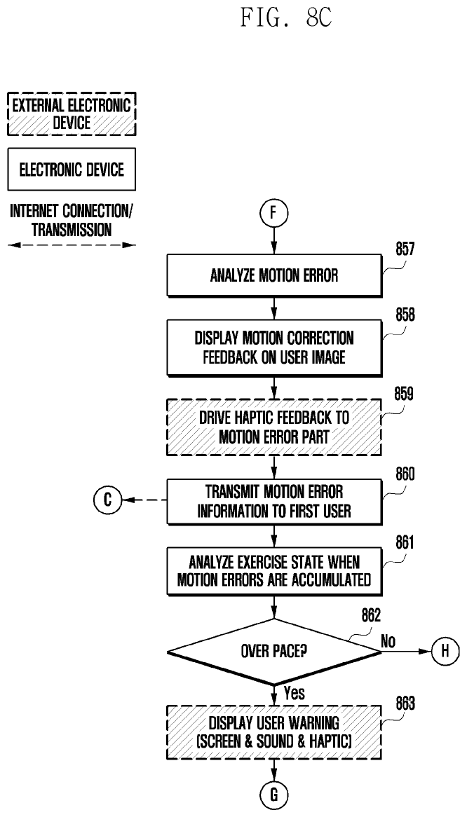

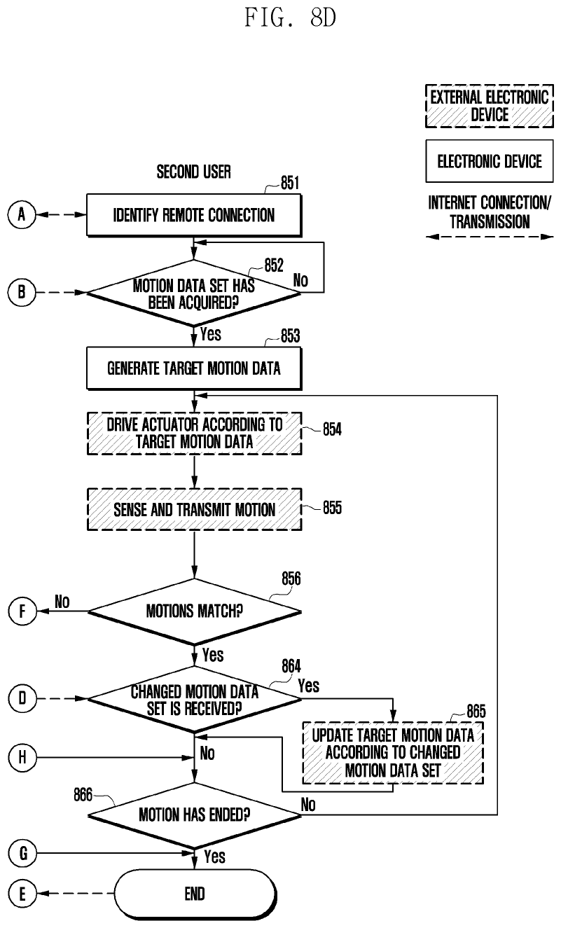

With reference to part (b) of FIG. 5B, a method of providing a motion-related content 512 and a motion data set may be in the form of being connected to a first user on the server and receiving a motion data set and a motion-related content 512 generated by the first user in real time. For example, the second user may access the server (e.g., application or site) on the electronic device 200 to be connected to the first user. The processor 220 may control the external electronic device 102 to display the motion-related content 512 including an image generated by the first user in real time on the display 160 of the electronic device 200 or to display the motion-related content 512 on the external electronic device 102 including the display. Further, the processor 220 may transmit a motion data set generated by the first user in real time and a target motion data set personalized to the second user generated based on second user information to the external electronic device 300 in real time. A specific embodiment related to this may be described in the description of FIG. 8A.

FIG. 5C is a diagram illustrating an example of an operation of generating a target motion data set based on a motion data set and user information by the processor 220 and transmitting the target motion data set to the external electronic device 300 according to various example embodiments.

According to an embodiment, the processor 220 may generate a target motion data set personalized to the user in a manner of converting a motion data set 424 including standard motion data 422, a load level 423, and the number of motions based on user information 521. For example, the processor 220 may correct a speed of the motion (e.g., a length of one cycle of the motion of the standard motion data 422) based on the length of the user's body part and/or the age of the user. For another example, the processor 220 may correct a load level based on the user's muscle strength level and/or health condition. For another example, the processor 220 may correct a motion trajectory (e.g., a motion trajectory range of the standard motion data 422) based on the user's flexibility and/or the user's age.

According to an embodiment, the processor 220 may transmit the target motion data set to the external electronic device 300 through the communication module 290. For example, the processor 220 may control the communication module 290 to transmit the target motion data set to the external electronic device 300 through wireless communication (e.g., Bluetooth, WiFi).

FIG. 51) is a diagram illustrating an example of an image including a user and feedback information generated by the processor 220 according to various example embodiments.

According to an embodiment, in the case that the difference between motion data and target motion data is greater than or equal to a designated value, the processor 220 may determine that the user is performing an erroneous motion. Further, the processor 220 may generate feedback information enabling the user to perform a target motion corresponding to the determination.

According to the embodiment of FIG. 5D, the processor 220 may display an image 561 including a user on the display 160 and/or the external electronic device including the display. For example, the processor 220 may acquire an image including a user taken by the camera 180 to display the image on the display 160 or the external electronic device. Further, the processor 220 may overlay and display image feedback 562 on the image 561. For example, the processor 220 may overlay and display the image feedback 562 at a position (e.g., left and right knee) of the user's body part requiring feedback on the image 561 in the form of a figure (e.g., below, a figure indicating rotation) and text (e.g., instruction phrases such as “lower your knees” and “rotate your knees”). Further, the processor 220 may display a feedback guide 564 in a portion of the image 561. For example, the feedback guide 564 may include a figure indicating a name of the motion and the degree of agreement with the target motion for each motion.

According to an embodiment, the processor 220 may transmit a signal for controlling the external electronic device 300 to provide haptic vibration to the external electronic device through the communication module 290. For example, the processor 220 may transmit a control signal to the external electronic device 300 so that the haptic sensor of the external electronic device 300 corresponding to the user's body part (e.g., left and right knee) requiring feedback generates haptic vibration 563.

FIG. 6A is a flowchart illustrating a method of generating target motion data personalized to a user with a method in which the processor (e.g., the processor 220 of FIG. 2) converts a motion data set based on user information according to various example embodiments.

According to various embodiments, in operation 610, the processor 220 may acquire a motion data set from the server.

According to an embodiment, the motion data set may include standard motion data related to other users having a standard body and/or additional information related to a level of difficulty of a motion input from a user in a manner of converting motion data related to a body motion based on a designated body.

According to an embodiment, the processor 220 may acquire a motion data set from the server through the communication module (e.g., the communication module 290 of FIG. 2). For example, the processor 220 may control the communication module 290 to download a motion data set from the server through wireless communication (e.g., WiFi, LTE).

According to various embodiments, in operation 620, the processor 220 may acquire user information from the memory (e.g., the memory 230 of FIG. 2).

According to an embodiment, the user information may include body information including a height and body part length, health information including age and health condition, and exercise information including a muscle strength level and flexibility.

According to an embodiment, the user information may be information input by a user or information generated by data accumulated in the memory 230.

According to various embodiments, in operations 630 to 660, the processor 220 may generate target motion data personalized to the user.

According to an embodiment, in operation 630, the processor 220 may compare user information with information of other users (hereinafter, referred to as standard users) having a standard body to calculate a difference between the information.

According to an embodiment, in operation 640, first information that the processor 220 compares may be a length of a lower limb and/or age. For example, the processor 220 may compare the length of the lower limb of the user with the length of the lower limb of the standard user and/or compare the age of the user with the age of the standard user. That is, the processor 220 may correct a length (speed) of one cycle of a unit motion based on a value obtained by subtracting the length of the standard user's lower limb from the length of the user's lower limb and/or a value obtained by subtracting the age of the standard user from the user's age.

In operation 641, the processor 220 may enlarge the length (speed) of one cycle of the unit motion corresponding to the user's lower limb length being longer than the standard user's lower limb length (in the case that the difference between the first information is greater than 0, operation 640>0). Further, in operation 642, the processor 220 may reduce the length (speed) of one cycle of the unit motion corresponding to the user's lower limb length being shorter than the standard user's lower limb length (in the case that the difference between the first information is smaller than 0, operation 640<0). Further, the processor 220 may not adjust the length (speed) of one cycle of the unit motion corresponding to the length of the user's lower limb being equal to the length of the lower limb of the standard user (in the case that the difference between the first information is 0, operation 640=0).

For example, the processor 220 may compare the age of the user with the age of the standard user, and in the case that the age of the user is greater than that of the standard user (in the case that the difference between the first information is greater than 0, operation 640>0), in operation 641, the processor 220 may enlarge the length (speed) of one cycle of the unit motion. Further, in operation 642, the processor 220 may reduce the length (speed) of one cycle of the unit motion corresponding to the age of the user being smaller than the age of the standard user (in the case that the difference between the first information is smaller than 0, operation 640<0). Further, the processor 220 may not adjust the length (speed) of one cycle of the unit motion corresponding to the age of the user being the same as that of the standard user (in the case that the difference between the first information is 0, operation 640=0).

According to an embodiment, in operation 650, third information that the processor 220 compares may be the difference in a flexibility factor and/or age. For example, the flexibility factor may be a numerical value of a range in which the body may move.

For example, the processor 220 may compare a user's flexibility factor with the standard user's flexibility factor and/or compare the user's age with the standard user's age. That is, the processor 220 may correct the range of the unit motion trajectory based on a value obtained by subtracting the standard user's flexibility factor from the user's flexibility factor and/or a value obtained by subtracting the standard user's age from the user's age.

For example, in operation 651, the processor 220 may enlarge the trajectory range of the motion corresponding to the user's flexibility factor being greater than the standard user's flexibility factor (in the case that the difference between the third information is greater than 0, operation 650>0). Further, in operation 652, the processor 220 may reduce the motion trajectory range corresponding to the user's flexibility factor being smaller than the standard user's flexibility factor (in the case that the difference between the third information is smaller than 0, operation 650<0). Further, in the case that the flexibility factor of the user is the same as that of the standard user (in the case that the difference between the third information is 0, operation 650=0), the processor 220 may not adjust the motion trajectory range.

For example, the processor 220 may compare the age of the user with the age of the standard user, and in operation 651, the processor 220 may enlarge the trajectory range of the unit motion corresponding to the age of the user being greater than that of the standard user (in the case that the difference between the third information is greater than 0, operation 650>0). Further, in operation 652, the processor 220 may reduce the trajectory range of the unit motion corresponding to the age of the user being smaller than that of the standard user (in the case that the difference between the third information is smaller than 0, operation 650<0). Further, in the case that the age of the user is equal to the age of the standard user (in the case that the difference between the third information is 0, operation 650=0), the processor 220 may not adjust the trajectory range of the unit motion.

According to an embodiment, in operation 660, the processor 220 may correct additional information including the load level and/or the number of motions based on the second information. For example, the second information may be the user's muscle strength level and/or health condition. For example, the processor 220 may compare the user's strength level with the standard user's strength level, and/or compare the user's health condition with the standard user's health condition. According to an embodiment, the muscle strength level may be a numerical value based on a load of a maximum or large value that the user may endure while performing a motion. According to an embodiment, the health condition may be a numerical value based on the maximum or high number of times when the user performs a motion.

For example, the processor 220 may compare the user's muscle strength level with the standard user's muscle strength level, and increase the load level and/or the number of motions corresponding to the user's muscle strength level being higher than the standard user's muscle strength level (in the case that the difference between the second information is greater than 0). Further, the processor 220 may lower the load level and/or the number of motions corresponding to the user's muscle strength level being lower than the standard user's muscle strength level the case that the difference between the second information is smaller than 0). Further, in the case that the user's muscle strength level is the same as the standard user's muscle strength level (in the case that the difference between the second information is 0), the processor 220 may not adjust the load level and/or the number of motions.

For example, the processor 220 may compare the user's health condition factor with the standard user's health condition factor and increase the load level and/or the number of motions corresponding to the user's health condition factor being higher than the standard user's health condition factor (in the case that the difference between the second information is greater than 0). Further, the processor 220 may lower the load level and/or the number of motions corresponding to the user's health condition factor being lower than the standard user's health condition factor (in the case that the difference between the second information is smaller than 0). Further, in the case that the user's health condition factor is the same as the standard user's health condition factor (in the case that the difference between the second information is 0), the processor 220 may not adjust the load level and/or the number of motions.

According to an embodiment, in operation 670, the processor 220 may generate a target motion data set including target exercise data in which motion data related to a body motion is personalized to the user and target additional data in which motion difficulty additional information (e.g., the load level of the motion and/or the number of repetitions of the motion) is personalized to the user.

FIG. 6B is a diagram illustrating an example in which the processor 220 generates target motion data personalized to a user in a manner of converting a motion data set based on user information, as illustrated in the flowchart of FIG. 6A according to various example embodiments.

With reference to part (a) of FIG. 6B, in the case that the difference between first information (e.g., lower limb length and/or age) is a positive (+) value, the processor 220 may enlarge a length (speed) of one cycle of the motion to correct motion data so as to slow down a speed performing a unit motion. Further, in the case that the difference between first information (e.g., lower limb length and/or age) is a negative (−) value, the processor 220 may reduce a length (speed) of one cycle of the motion to correct motion data so as to speed up a speed performing the unit motion.

With reference to part (b) of FIG. 6B, in the case that the difference between second information (e.g., muscle strength level and/or health condition) is a positive (+) value, the processor 220 may correct additional information so as to increase the load level. Further, in the case that the difference between second information (e.g., muscle strength level and/or health condition) is a negative (−) value, the processor 220 may correct additional information so as to lower the load level.

With reference to part (c) of FIG. 6B, in the case that the difference between third information (e.g., flexibility factor and/or age) is a positive (+) value, the processor 220 may enlarge the motion trajectory range to correct motion data. Further, in the case that the difference between third information (e.g., flexibility factor and/or age) is a negative (−) value, the processor 220 may reduce the motion trajectory range to correct motion data.

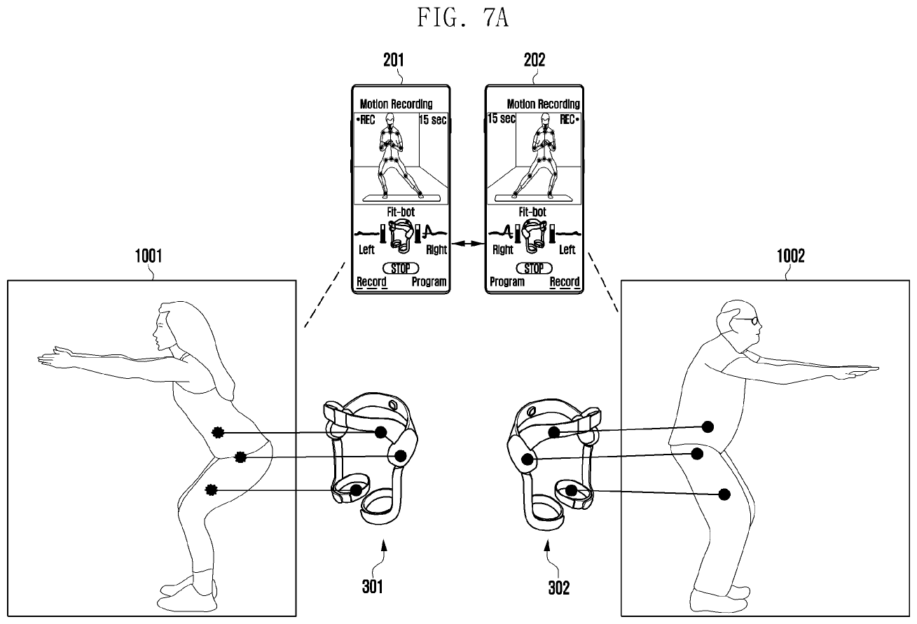

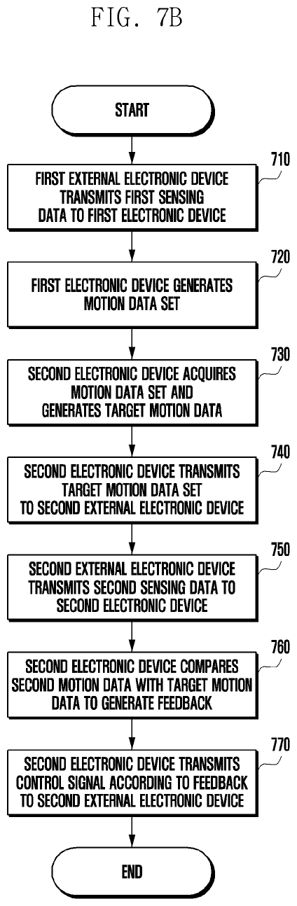

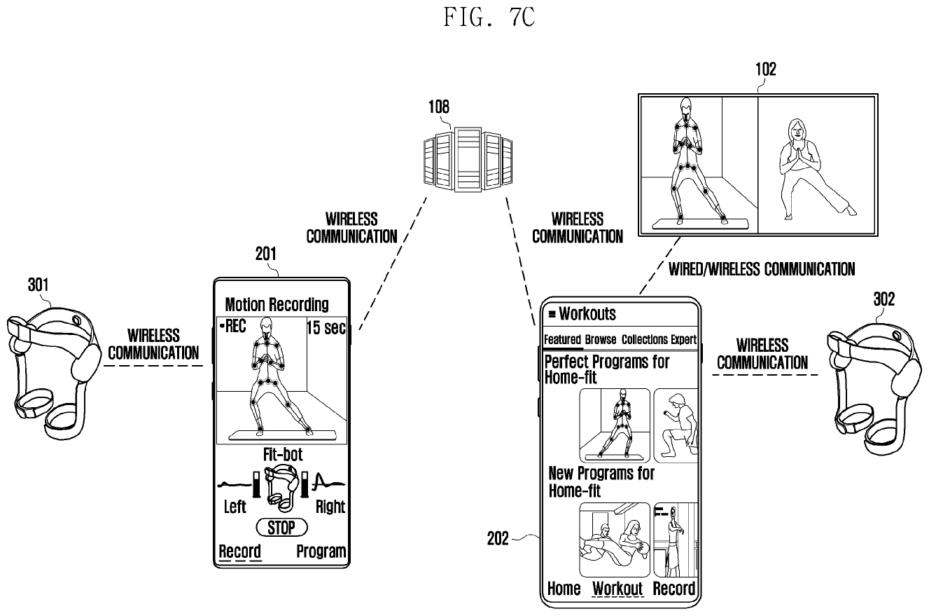

FIG. 7A is a diagram illustrating an example of a constitution of a motion assistance system according to various example embodiments.

According to various embodiments, the motion assistance system may include any one of a first external electronic device 301 worn by a first user 1001, a second external electronic device 302 worn by a second user 1002, a first electronic device 201 used by the first user 1001, or a second electronic device 202 used by the second user 1002.

FIG. 7B is a flowchart illustrating an operation in which a motion assistance system assists a user's motion according to an embodiment.

According to various embodiments, the motion assistance system may include at least one of a first electronic device 201, a second electronic device 202, a first external electronic device 301, a second external electronic device 302, or a server (e.g., a server 108 of FIG. 7C).

According to various embodiments, in operation 710, the first external electronic device 301 may transmit first sensing data to the first electronic device 201.

According to an embodiment, first sensing data may include a value related to a motion of a body (e.g., an inclined angle and/or a moved angle of the body) of the first user 1001 wearing the first external electronic device 301 measured by various sensors (e.g., the first sensor 310 and/or the second sensor 320 of FIG. 3) of the first external electronic device 301.

According to various embodiments, in operation 720, the first electronic device 201 may generate a motion data set.

According to an embodiment, the first electronic device 201 may generate a motion data set including standard motion data related to other users having a standard body and/or additional information related to a level of difficulty of a motion input from the first user 1001 in a manner of converting the first motion data based on a designated body.

According to an embodiment, the first electronic device 201 may generate first motion data related to a motion of the body based on first sensing data. For example, the first electronic device 201 may generate first motion data (e.g., a sequential motion trajectory of the body) based on a value measured by the first sensor 310 and/or a value measured by the second sensor 320 included in the first sensing data. According to an embodiment, the first electronic device 201 may analyze motion data related to multiple times of motion to distinguish motions constituting one cycle (one time). The first electronic device 201 may divide first motion data into one cycle of a motion and calculate an average of values in one cycle to generate average motion data. The average motion data may include a value related to a motion in one cycle (e.g., motion trajectory range and/or motion execution speed).

According to an embodiment, the first electronic device 201 may store a user's body information (e.g., body height, body part length, flexibility factor, health condition factor, muscle strength level, and age).

According to an embodiment, the first electronic device 201 may generate standard motion data related to other users having a standard body in a manner of converting average motion data based on designated body information. For example, standard body information may be information including various information (e.g., height, length of body part, flexibility factor, health condition factor, muscle strength level, and age of a standard body based on male and female standard body (e.g., ISO 7250)) of a designated body. Standard body information is body information stored by the first electronic device 201, and the first electronic device 201 may convert the standard body information into standard motion data based on a comparison result between the user's body information and the standard body information. For example, the first electronic device 201 may normalize average motion data to generate standard motion data (e.g., motion trajectory range and/or motion execution speed) in a manner of comparing the standard body information and the body information of the first user 1001 and correcting average motion data corresponding to the difference between body information.

According to an embodiment, the first electronic device 201 may receive an input of additional information related to a level of difficulty of the motion from the first user 1001. For example, the additional information may include a load level of a motion and/or the repetition number of a motion. According to an embodiment, the first electronic device 201 may display a screen for a user input on the display (e.g., the display module 160 of FIG. 1, comprising a display) and receive an input of additional information through the screen.

According to an embodiment, the first electronic device 201 may transmit the motion data set to the server through the communication module 290 comprising communication circuitry. For example, the first electronic device 201 may control the communication module 290 to transmit the motion data set to the server through wireless communication (e.g., internet communication such as WiFi or LTE).

According to an embodiment, the first electronic device 201 may acquire a motion-related content and transmit the motion-related content to the server through the communication module 290 together with a motion data set. For example, the motion-related content may include an image including the first user 1001 performing the motion. For example, a motion included in the motion data set may be the same motion as that included in the motion-related content. According to an embodiment, the first electronic device 201 may acquire a motion-related content through a camera (e.g., the camera module 180 of FIG. 1). For example, the first electronic device 201 may acquire an image of the first user 1001 performing a motion taken by the camera 180. According to an embodiment, the first electronic device 201 may acquire a content related to a motion from the server. For example, the first electronic device 201 may download an image including the first user 1001 performing a motion from the server.

According to various embodiments, in operation 730, the second electronic device 202 may acquire a motion data set and generate target motion data.

According to an embodiment, the second electronic device 202 may acquire a motion data set from the server through the communication module (e.g., the communication module 290 of FIG. 2, comprising communication circuitry). For example, the second electronic device 202 may control the communication module 290 to download a motion data set from the server through wireless communication (e.g., WiFi, LTE).

According to an embodiment, the second electronic device 202 may acquire a motion related content from the server through the communication module 290 together with the motion data set. For example, the motion-related content may be an image including the first user 1001 performing the motion. For example, a motion included in the motion data set may be the same motion as that included in the motion-related content.

According to an embodiment, the motion related content and the motion data set may be in the form of downloading a file uploaded on the server. For example, the second user 1002 may download a motion data set or download together a content related to the motion and a motion data set while accessing a server (e.g., application, site) on the second electronic device 202, selecting a content related to a motion to be performed, and streaming a content related to the motion.

According to an embodiment, the motion related content and the motion data set may be in the form of being connected to the first user 1001 on the server to receive a motion data set and a motion related content generated by the first user 1001 in real time. For example, the second user 1002 may access the server (e.g., application, site) on the second electronic device 202 to be connected to the first user 1001, and receive an image including the first user 1001 and a motion data set generated by the first user 1001 using the first external electronic device 301.

According to an embodiment, the second electronic device 202 may acquire second user information from the memory (e.g., the memory 230 of FIG. 2).

According to an embodiment, the second user information may include at least one of body information including a height and body part length, health information including age and health condition, or exercise information including a muscle strength level and flexibility.

According to an embodiment, the second user information may be information input by the second user 1002 or information generated by data accumulated in the memory 230.

According to various embodiments, the second electronic device 202 may generate a target motion data set personalized to the second user 1002.

According to an embodiment, the target motion data set may include target motion data personalized to the second user and/or target additional information personalized to the second user in a manner of converting based on second user information.

According to an embodiment, the second electronic device 202 may generate target motion data personalized to the second user 1002 in a manner of converting motion data based on second user information. For example, the second electronic device 202 may adjust a motion speed based on the body part length and/or the age of the second user 1002. For another example, the second electronic device 202 may adjust a load level based on the muscle strength level and/or the health condition of the second user 1002. As another example, the second electronic device 202 may correct a motion trajectory range based on the flexibility and/or age of the second user 1002.

According to an embodiment, the second electronic device 202 may generate target additional information personalized to the second user 1002 in a manner of converting additional information based on second user information. For example, the second electronic device 202 may adjust the load level based on the muscle strength level and/or the health condition of the second user 1002.

According to various embodiments, in operation 740, the second electronic device 202 may transmit the target lotion data set to the second external electronic device 302.

According to an embodiment, the second electronic device 202 may transmit the target motion data set to the second external electronic device 302 through the communication module 290. For example, the second electronic device 202 may control the communication module 290 to transmit a target motion data set to the second external electronic device 302 through wireless communication (e.g., Bluetooth, WiFi).

According to an embodiment, the second external electronic device 302 may control an actuator (e.g., the actuator 330 of FIG. 3) based on the target motion data set. For example, by adjusting the torque strength and direction of the actuator 330 based on the target motion data set, the second external electronic device 302 may assist the second user 1002 to perform the target motion.

According to various embodiments, in operation 750, the second external electronic device 302 may transmit second sensing data to the second electronic device 202.

According to an embodiment, the sensing data may include a value related to a motion of the body (e.g., an inclined angle and/or a moved angle of the body) of the second user 1002 wearing the second external electronic device 302 measured by various sensors (e.g., the first sensor 310 and/or the second sensor 320 of FIG. 3) of the second external electronic device 302.

According to an embodiment, the second electronic device 202 may acquire second sensing data from the second external electronic device 302 through the communication module 290. For example, the second electronic device 202 may acquire second sensing data received from the second external electronic device 302 by the communication module 290 through wireless communication (e.g., short-range communication such as Bluetooth or WiFi).

According to an embodiment, the processor 220 may generate second motion data related to a motion of the body based on the second sensing data. For example, the processor 220 may generate second motion data (e.g., sequential motion trajectory) based on a value measured by the first sensor 310 and/or a value measured by the second sensor 320 included in the second sensing data.

According to various embodiments, in operation 760, the second electronic device 202 may compare the second motion data with the target motion data to generate feedback.

According to an embodiment, in the case that an absolute value of the difference between the second motion data and the target motion data is greater than or equal to a designated value, the second electronic device 202 may determine that the second user is performing an erroneous motion. Further, the second electronic device 202 may generate feedback information for guiding the second user to perform the target motion corresponding to the determination. For example, the feedback information may be in various forms such as image feedback, haptic feedback, and voice feedback.

According to an embodiment, the second electronic device 202 may display an image including a second user on the display (e.g., the display module 160 of FIG. 1) and/or an external electronic device including the display. For example, the second electronic device 202 may acquire an image including the second user taken by a camera (e.g., the camera module 180 of FIG. 1) and display the image on the display 160 or the external electronic device. Further, the second electronic device 202 may overlay and display image feedback on the image. For example, the second electronic device 202 may overlay and display image feedback at a position of the body part of the second user 1002 requiring feedback in the form of a figure or text on an image including the second user 1002.

According to an embodiment, the second electronic device 202 may output audio feedback to an external electronic device including an audio module and/or an audio device (e.g., the audio module 170 of FIG. 1).

According to various embodiments, in operation 770, the second electronic device 202 may transmit a control signal according to feedback to the second external electronic device 302.

According to an embodiment, the second electronic device 202 may transmit a signal for controlling the second external electronic device 302 to generate haptic vibration based on feedback information to the second external electronic device through the communication module 290. For example, the second electronic device 202 may transmit a control signal to the second external electronic device 302 so that a haptic sensor of the second external electronic device 302 corresponding to a body part of the second user 1002 requiring feedback vibrates.

FIG. 7C is a diagram illustrating an example of connection between components of a motion assistance system according to various example embodiments.

According to various embodiments, the motion assistance system may include any one of a first external electronic device 301, a second external electronic device 302, a first electronic device 201, a second electronic device 202, a server 108, or an external display device 102.

According to an embodiment, the first external electronic device 301 and the first electronic device 201 may transmit and receive information to and from each other through short-range wireless communication (e.g., Bluetooth, WiFi).

According to an embodiment, the first electronic device 201 may transmit and receive information to and from the server 108 through wireless communication (e.g., Internet communication such as LTE and WiFi).

According to an embodiment, the second electronic device 202 may transmit and receive information to and from the server 108 through wireless communication (e.g., Internet communication such as LTE and WiFi).

According to an embodiment, the second electronic device 202 and the display device 102 may transmit and receive information to and from each other through short-range wireless communication (e.g., Bluetooth, WiFi) and/or wired communication.

According to an embodiment, the second electronic device 202 and the second external electronic device 302 may transmit and receive information to and from each other through short-range wireless communication (e.g., Bluetooth,

FIG. 8A is a diagram illustrating an example in which a first user and a second user perform a motion assistance motion in real time in a motion assistance system according to various example embodiments.

According to an embodiment, a first electronic device (e.g., the electronic device 201 of FIG. 7B) may be connected to a second electronic device (e.g., the electronic device 202 of FIG. 7B) through at least the server 10S. Further, the first user 1001 may perform a motion by wearing the first external electronic device 301. Further, the first external electronic device 301 and the first electronic device 201 may transmit and receive information to and from each other through short-range wireless communication (e.g., Bluetooth, WiFi).

According to an embodiment, the second electronic device 202 may be connected to the first electronic device 201 through at least the server 108. Further, the second electronic device 202 and the second external electronic device 302 may transmit receive information to and from each other through short-range wireless communication (e.g., Bluetooth, WiFi). Further, the second user 1002 may perform a motion by wearing the second external electronic device 302.

According to an embodiment, the first electronic device 201 and/or the second electronic device 202 may display a first user screen (e.g., motion related content 512) and/or a second user screen (e.g., an image 561 including a second user).

FIGS. 8B, 8C, and 8D are a flowchart illustrating a method in which a first external electronic device (e.g., the first external electronic device 301 of FIG. 7A), a second external electronic device (e.g., the second electronic device 302 of FIG. 7A), a first electronic device (e.g., the first electronic device 201 of FIG. 7A), and a second electronic device (e.g., the second electronic device 202 of FIG. 7A) assist a user's motion in real time by interacting with each other in a motion assistance system according to various example embodiments.

According to various embodiments, operations 801 to 814 may be operations performed by the first electronic device 201 and the first external electronic device 301, and operations 851 to 867 may be an operation performed by the second electronic device 202 and the second external electronic device 302.

According to various embodiments, in operation 801, the first electronic device 201 may identify a remote connection with the second electronic device 202. According to an embodiment, the first electronic device 201 and the second electronic device 202 may transmit and receive information to and from each other through the server (e.g., the server 108 of FIG. 7C).

According to various embodiments, in operation 802, the first electronic device 201 may start generating first motion data. According to an embodiment, the first motion data is data related to a motion of a body of the first user 1001 and may include a sequential motion trajectory of the body.

According to various embodiments, in operation 803, the first external electronic device 301 may transmit first sensing data obtained by sensing the motion of the first user 1001 to the first electronic device 201.

According to an embodiment, the first sensing data may include a value (e.g., an inclined angle and/or a moved angle of the body) related to a motion of the body of the first user 1001 wearing the first external electronic device 301 measured by various sensors (e.g., the first sensor 310 and/or the second sensor 320 of FIG. 3) of the first external electronic device 301.

According to various embodiments, in operation 804, the first electronic device 201 may determine whether generation of the first motion data has ended, and repeatedly perform operation 803 until generation of the first motion data has ended.

According to various embodiments, in operation 805, the first electronic device 201 may generate a motion data set.

According to an embodiment, the first electronic device 201 may generate a motion data set including standard motion data related to other users having a standard body and/or additional information related to a level of difficulty of a motion input from the first user 1001 in a manner of converting first motion data based on a designated body.

According to an embodiment, the first electronic device 201 may generate first motion data related to a motion of the body based on the first sensing data. For example, the first electronic device 201 may generate first motion data (e.g., sequential motion trajectory of the body) based on a value measured by the first sensor 310 and/or a value measured by the second sensor 320 included in the first sensing data. According to an embodiment, the first electronic device 201 may analyze motion data related to multiple times of motion to distinguish motions constituting one cycle (one time). The first electronic device 201 may divide motion data into one cycle of a motion and calculate an average of values in one cycle to generate average motion data. The average motion data may include a value related to a motion in one cycle (e.g., motion trajectory range and/or motion execution speed).

According to an embodiment, the first electronic device 201 may store a user's body information (e.g., body height, body part length, flexibility factor, health condition factor, muscle strength level, and age).

According to an embodiment, the first electronic device 201 may generate standard motion data related to other users having a standard body in a manner of converting average motion data based on designated body information. For example, standard body information may be information including various information (e.g., height, length of body part, flexibility factor, health condition factor, muscle strength level, and age of a standard body based on male and female standard body (e.g., ISO 7250)) of a designated body. Standard body information is body information stored by the first electronic device 201, and the first electronic device 201 may convert standard body information into standard motion data based on a comparison result between the user's body information and the standard body information. For example, the first electronic device 201 may normalize average motion data to generate standard motion data (e.g., motion trajectory range and/or motion execution speed) in a manner of comparing the standard body information with the users body information and correcting average motion data corresponding to the difference between body information.

According to an embodiment, the first electronic device 201 may receive an input of additional information related to a level of difficulty of a motion from the first user 1001. For example, the additional information may include a load level of a motion and/or the repetition number of a motion. According to an embodiment, the first electronic device 201 may display a screen for user input on the display (e.g., the display module 160 of FIG. 1) and receive an input of additional information through the screen.

According to various embodiments, in operation 806, the first electronic device may transmit a motion data set to the According to an embodiment, the first electronic device 201 may transmit the motion data set to the server through the communication module 290. For example, the first electronic device 201 may control the communication module 290 to transmit the motion data set to the server 108 through wireless communication (e.g., internet communication such as WiFi or LTE).

According to various embodiments, in operation 807, the first electronic device 201 may determine whether user motion error information has been received. According to an embodiment, the user motion error information may include information related to a generated error corresponding to the second electronic device 202 determining that there is an error in the motion of the second user 1002 in operation 860. According to an embodiment, the first electronic device 201 may receive user motion error information transmitted from the second electronic device 202 to the server from the server.

According to various embodiments, in operation 808, the first electronic device 201 may display feedback for correcting the motion on the user image corresponding to receiving user motion error information.

According to an embodiment, the first electronic device 201 may display an image 561 including a second user on the display (e.g., the display module 160 of FIG. 1) and/or the external electronic device including the display. For example, the first electronic device 201 may acquire an image 561 including the second user 1002 taken by a camera (e.g., the camera module 180 of FIG. 1) of the second electronic device 202 to display the image 561 on the display 160 or the external electronic device. Further, the first electronic device 201 may overlay and display image feedback on the image 561. For example, the first electronic device 201 may overlay and display image feedback at a position of a body part of the second user 1002 requiring feedback in the form such as a figure or text on the image 561 including the second user 1002.

According to various embodiments, in operation 809, the first external electronic device 301 may drive haptic feedback to a motion error part of the second user 1002.

According to an embodiment, the first external electronic device 301 may receive a signal controlling to generate haptic vibration from the first electronic device 201. For example, the first external electronic device 301 may receive, from the first electronic device 201, a signal controlling to vibrate a haptic sensor corresponding to a body part in which a motion error of the second user 1002 has occurred.

According to various embodiments, in operation 810, the first electronic device 201 may analyze a state of the second user 1002 corresponding to accumulation of motion errors of the second user 1002.

According to various embodiments, in operation 811, the first electronic device may display the state of the second user 1002. According to an embodiment, the state of the second user 1002 may include motion error information including the number of motion errors and a motion error rate of the second user 1002.

According to various embodiments, in operation 812, the first electronic device 201 may determine whether there is a motion data set change input from the first user 1001. According to an embodiment, the first user 1001 may change additional information including a load level and/or the number of motions in the first electronic device 201.

According to various embodiments, in operation 813, the first electronic device 201 may change a motion data set and transmit the changed motion data set to the server corresponding to a motion change input (operation 812—Yes).

According to various embodiments, the first electronic device 201 may determine whether the motion has ended corresponding to the absence of a motion change input (operation 812—No). According to various embodiments, in operation 814, the first electronic device 201 may repeatedly perform operations 807 to 813 until it is determined that the motion has ended.

According to various embodiments, in operation 851, the second electronic device 202 may identify a remote connection with the first electronic device 201. According to an embodiment, the second electronic device 202 and the first electronic device 201 may transmit and receive information to and from each other through the server.

According to various embodiments, in operation 852, the second electronic device 202 may determine whether a motion data set has been acquired. According to an embodiment, the second electronic device 202 may acquire a motion data set from the server. According to an embodiment, the motion data set may be transmitted from the first electronic device 201 to the server in operation 806.

According to various embodiments, the second electronic device 202 may repeatedly perform operation 852 until a motion data set is acquired.

According to various embodiments, in operation 853, the second electronic device 202 may generate target motion data corresponding to acquiring the motion data set (operation 852—Yes).

According to an embodiment, the second electronic device 202 may acquire second user information from the memory (e.g., the memory 230 of FIG. 2). According to an embodiment, the second user information may include at least one of body information including a height and body part length, health information including age and health condition, or exercise information including a muscle strength level and flexibility of the second user 1002. According to an embodiment, the user information may be information input by the second user 1002 or information generated by data accumulated in the memory 230. According to various embodiments, the second electronic device 202 may generate a target motion data set personalized to the second user 1002. According to an embodiment, the target motion data set may include target motion data and/or target additional information personalized to the second user in a manner of converting based on second user information.

According to an embodiment, the second electronic device 202 may generate target motion data personalized to the second user 1002 in a manner of converting the motion data set based on the second user information. For example, the second electronic device 202 may adjust a motion speed based on the body part length and/or the age of the second user 1002. For another example, the second electronic device 202 may adjust a load level based on the muscle strength level and/or health condition of the second user 1002. As another example, the second electronic device 202 may correct a motion trajectory range based on flexibility and/or age of the second user 1002. According to an embodiment, the second electronic device 202 may generate target additional information personalized to the second user 1002 in a manner of converting additional information based on second user information. For example, the second electronic device 202 may adjust the load level based on the muscle strength level and/or health condition of the second user 1002.

According to various embodiments, in operation 854, the second external electronic device 302 may drive an actuator (e.g., the actuator 330 of FIG. 3) according to a target motion data set.

According to an embodiment, the second external electronic device 302 may receive a target motion data set from the second electronic device 202. According to an embodiment, the second external electronic device 302 may control an actuator (e.g., the actuator 330 of FIG. 3) based on a target motion data set. For example, the second external electronic device 302 may adjust the torque intensity and direction of the actuator 330 based on the target motion data set to assist the second user 1002 to perform the target motion.

According to various embodiments, in operation 855, the second external electronic device 302 may measure the motion of the second user 1002 and transmit the measured second sensing data to the second electronic device 202.

According to an embodiment, the second external electronic device 302 may transmit second sensing data to the second electronic device 202 through the communication module. According to an embodiment, the second sensing data may include a value (e.g., an inclined angle and/or a moved angle of the body) related to a motion of the body of the second user 1002 wearing the second external electronic device 302 measured by various sensors (e.g., the first sensor 310 and/or the second sensor 320 of FIG. 3) of the second external electronic device 302.

According to various embodiments, in operation 856, the second electronic device 202 may determine whether a motion sensed by the second external electronic device 302 matches a target motion.

According to an embodiment, the processor 220 may generate second motion data related to a motion of the body based on the second sensing data. For example, the processor 220 may generate second motion data (e.g., sequential motion trajectory) based on a value measured by the second sensor 310 and/or a value measured by the second sensor 320 included in the second sensing data. According to an embodiment, in the case that an absolute value of the difference between the second motion data and the target motion data is greater than or equal to a designated value, the second electronic device 202 may determine that the second user is performing an erroneous motion.

According to various embodiments, in operation 857, the second electronic device 202 may analyze a motion error corresponding to the motion not matching (operation 856—No).

According to an embodiment, the second electronic device 202 may analyze a motion error to generate feedback information.

According to various embodiments, in operation 858, the second electronic device 202 may display motion feedback on the image 561 including the second user.

According to an embodiment, the second electronic device 202 may display an image 561 including the second user on the display (e.g., the display module 160 of FIG. 1) and/or the external electronic device including the display. For example, the second electronic device 202 may acquire an image 561 including the second user 1002 taken by a camera (e.g., the camera module 180 of FIG. 1) thereof to display the image 561 on the display 160 or the external electronic device. Further, the second electronic device 202 may overlay and display image feedback on the image 561 including the second user. For example, the second electronic device 202 may overlay and display image feedback at a position of a body part of the second user 1002 requiring feedback in the form of a figure or text on the image 561 including the second user 1002.

According to various embodiments, in operation 859, the second external electronic device 302 may drive haptic feedback to a motion error part.

According to an embodiment, the second external electronic device 302 may receive a signal controlling to generate haptic vibration from the second electronic device 202. For example, the second external electronic device 302 may receive, from the second electronic device 202, a signal controlling to vibrate a haptic sensor corresponding to a body part in which a motion error of the second user 1002 has occurred.

According to various embodiments, in operation 860, the second electronic device 202 may transmit motion error information to the first electronic device 201 through the server.

According to various embodiments, in operation 861, the second electronic device 202 may analyze a state of the second user 1002 corresponding to accumulation of motion errors. According to an embodiment, the state of the second user 1002 may include motion error information including the number of motion errors and a motion error rate of the second user 1002.

According to various embodiments, in operation 862, the second electronic device 202 may determine whether the motion of the second user 1002 is over pace. According to an embodiment, the second electronic device 202 may determine that the motion of the second user 1002 is over pace corresponding to the number of motion errors and the ratio of motion errors of the second user 1002 being greater than or equal to a designated value.

According to various embodiments, in operation 863, the second external electronic device 302 may display a user warning corresponding to the second electronic device 202 determining that the motion of the second user 1002 is over pace. The user warning may be various forms such as haptic vibration, screen display, and voice display. According to an embodiment, the motion assistance system may end the motion corresponding to operation 863 being performed.

According to various embodiments, in operation 865, the second external electronic device 302 may update a target motion data set based on the changed motion data set corresponding to the second electronic device 202 not determining that the user's motion is over pace in operation 862 and/or determining that the motions match in operation 856, and receiving the changed motion data set in operation 864.

According to various embodiments, in operation 866, the second electronic device 202 may determine whether the motion has ended. According to various embodiments, in operation 814, the second electronic device 202 may repeatedly perform operations 854 to 865 until it is determined that the motion has ended.

According to various example embodiments, an electronic device may include a communication module comprising communication circuitry; and a processor operatively connected, directly or indirectly, to the communication module, wherein the communication module may be configured to communicate with a server and an external electronic device including a sensor configured to measure a user's motion, wherein the processor may be configured to control to acquire sensing data obtained by measuring a user's motion from the external electronic device through the communication module, to generate a motion data set including standard motion data related to other users having a standard body in a manner of converting motion data including a sequential motion trajectory of the body based on the sensing data based on information of the user, and to transmit the motion data set to the server through the communication module.

According to various example embodiments, the processor may be configured to receive a user input for additional information related to a level of difficulty of the motion, and to add the additional information to the motion data set.

According to various example embodiments, the processor may be configured to generate standard motion data using an average value of the sensing data obtained by sensing multiple times of motion of the user.

According to various example embodiments, the processor may be configured to control to acquire contents related to a motion including the same motion as the motion data set, and to transmit the motion data set and contents related to the motion to the server through the communication module.

According to various example embodiments, the motion data set may include information related to a motion included in contents related to the motion.

According to various example embodiments, the electronic device may further include a camera, wherein the processor may be configured to acquire contents related to the motion including an image taken through the camera.

According to various example embodiments, an electronic device may include a communication module; a memory configured to store user information; and a processor operatively connected to the communication module and the memory, wherein the communication module may be configured to communicate with a server and an external electronic device including a sensor and an actuator, wherein the processor may be configured to receive a motion data set including standard motion data related to motions of other users having a standard body from the server through the communication module, to acquire user information stored in the memory, to generate a target motion data set personalized to a user in a manner of converting the motion data set based on the user information, to acquire sensing data obtained by measuring a user's motion from the external electronic device through the communication module, to generate feedback by comparing motion data including a sequential motion trajectory of a body based on the sensing data with the target motion data set, and to transmit a signal for controlling the external electronic device based on the feedback to the external electronic device through the communication module.

According to various example embodiments, the electronic device may be configured to store the user information including at least one of the user's body information, health information, or exercise information, wherein the processor may be configured to acquire the motion data set including at least one of a motion speed, a trajectory range of a motion, a load level, or the number of motions, and to generate the target motion data set by correcting at least one of a motion speed, a motion trajectory, a load level, or the number of motions based on at least one of the user's body information, health information, or exercise information.

According to various example embodiments, the electronic device may further include a camera; and a display, wherein the camera may be configured to take an image including a user, wherein the processor may be configured to display the feedback and the image on the display.

According to various example embodiments, the processor may be configured to transmit a signal for controlling the external electronic device to vibrate based on the feedback to the external electronic device through the communication module.

According to various example embodiments, the electronic device may further include a display, wherein the processor may be configured to acquire contents related to a motion including the same motion as that of the motion data set from the server through the communication module, and to synchronize the feedback and contents related to the motion and to output the feedback and the contents on the display.

According to various example embodiments, a motion assistance system may include a first wearable device including a first sensor; and a second wearable device including a second sensor and an actuator, wherein the first wearable device may be configured to acquire first motion data related to a motion of a first user measured by the first sensor, wherein the second wearable device may be configured to acquire the first motion data, to acquire second motion data related to a motion of a second user measured by the first sensor, and to control the actuator based on a result of comparing the first motion data and the second motion data.

According to various example embodiments, the notion assistance system may further include a server, wherein the first wearable device may be configured to transmit the first motion data to the server, and the second wearable device may be configured to receive the first motion data from the server.

According to various example embodiments, in the motion assistance system, the first wearable device may be configured to store information of a first user and to generate standard motion data in a manner of converting the first motion data to be related to another user having a standard body based on information of the first user.

According to various example embodiments, the second wearable device may be configured to store information of a second user, to acquire the standard motion data, to generate target motion data personalized to the second user in a manner of converting the standard motion data based on information of the second user, and to control the actuator based on the target motion data. “Based on” as used herein covers based at least on.

According to various example embodiments, the motion assistance system may further include a first electronic device, and the first electronic device may be configured to perform some of motions of the first wearable device.

According to various example embodiments, the motion assistance system may further include a second electronic device, and the second electronic device may be configured to perform some of motions of the second wearable device.

According to various example embodiments, the motion assistance system may further include a first electronic device and a second electronic device, wherein the first electronic device may be configured to acquire feedback from the second user to output feedback on a display, and the second electronic device may be configured to acquire an exercise guide of the first user and to output the exercise guide on the display.

It should be appreciated that various embodiments and the terms used therein are not intended to limit the technological features set forth herein to particular embodiments and include various changes, equivalents, or replacements for corresponding embodiment.

With regard to the description of the drawings, similar reference numerals may be used to refer to similar or related elements, it is to be understood that a singular form of a noun corresponding to an item may include one or more of the things, unless the relevant context clearly indicates otherwise.

As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” “communicatively”, as “coupled with,” “coupled to,” “connected with,” or “connected to” another element (e.g., a second element), it denotes that the element may be coupled with the other element directly (e.g., wiredly), wirelessly, or via at least a third element(s).

While the disclosure has been illustrated and described with reference to various embodiments, it will be understood that the various embodiments are intended to be illustrative, not limiting. It will further be understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

1. An electronic device, comprising:
a communication module comprising communication circuitry; and
a processor operatively connected to the communication module,
wherein the communication module is configured to communicate with a server and an external electronic device including a sensor configured to measure a user's motion,
wherein the processor is configured to control to:
acquire sensing data related to the user's motion from the external electronic device through at least the communication module,
generate a motion data set including reference motion data related to other users having a reference body in a manner of converting motion data including a sequential motion trajectory of a body based on the sensing data based on information of the user, and
transmit the motion data set to the server through at least the communication module.

2. The electronic device of claim 1, wherein the processor is configured to:
receive a user input for additional information related to a level of difficulty of the motion, and
add the additional information to the motion data set.

3. The electronic device of claim 1, wherein the processor is configured to generate the reference motion data using at least an average value of the sensing data obtained by sensing multiple times of motion of the user.

4. The electronic device of claim 1, wherein the processor is configured to control to:
acquire contents related to a motion including the same motion as that of the motion data set, and
transmit the motion data set and the contents related to the motion to the server through at least the communication module.

5. The electronic device of claim 4, wherein the motion data set comprises information related to a motion included in the contents related to the motion.

6. The electronic device of claim 4, further comprising a camera,
wherein the processor is configured to acquire contents related to the motion including an image taken through the camera.

7. An electronic device, comprising:
a communication module comprising communication circuitry;
a memory configured to store user information; and
a processor operatively connected to the communication module and the memory,
wherein the communication module is configured to communicate with a server and an external electronic device including a sensor and an actuator,
wherein the processor is configured to:
receive a motion data set including reference motion data related to motions of other users having a reference body from the server through at least the communication module,
acquire user information stored in the memory,
generate a target motion data set personalized to a user in a manner of converting the motion data set based on the user information,
acquire sensing data related to a user's motion from the external electronic device through at least the communication module,
generate feedback at least by comparing motion data including a sequential motion trajectory of a body based on the sensing data with the target motion data set, and
transmit a signal for controlling the external electronic device based on the feedback to the external electronic device through at least the communication module.

8. The electronic device of claim 7, wherein the electronic device is configured to store the user information including at least one of the user's body information, health information, or exercise information,
wherein the processor is configured to:
acquire the motion data set including at least one of: a motion speed, a trajectory range of a motion, a load level, or the number of motions, and
generate the target motion data set at least by correcting at least one of: a motion speed, a motion trajectory, a load level, or the number of motions based on at least one of: the user's body information, health information, or exercise information.

9. The electronic device of claim 7, further comprising:
a camera; and
a display,
wherein the camera is configured to take an image including a user, and
the processor is configured to display the feedback and the image on the display.

10. The electronic device of claim 7, wherein the processor is configured to control to transmit a signal for controlling the external electronic device to vibrate based on the feedback to the external electronic device through at least the communication module.

11. The electronic device of claim 7, further comprising a display,
wherein the processor is configured to:
acquire contents related to a motion including the same motion as that of the motion data set from the server through at least the communication module, and
synchronize the feedback and the contents related to the motion and output the feedback and the contents on the display.

12. A motion assistance system, comprising:
a first wearable device including a first sensor; and
a second wearable device including a second sensor and an actuator,
wherein the first wearable device is configured to acquire first motion data related to a motion of a first user measured by the first sensor,
wherein the second wearable device is configured to:
acquire the first motion data,
acquire second motion data related to a motion of a second user measured by the first sensor, and
control the actuator based on a result of comparing the first motion data and the second motion data.

13. The motion assistance system of claim 12, further comprising a server,
wherein the first wearable device is configured to transmit the first motion data to the server, and
the second wearable device is configured to receive the first motion data from the server.

14. The motion assistance system of claim 12, wherein the first wearable device is configured to:
store information of the first user, and
generate reference motion data in a manner of converting the first motion data to be related to other user having a reference body based on the information of the first user.

15. The motion assistance system of claim 14, wherein the second wearable device is configured to:
store information of the second user,
acquire the reference motion data,
generate target motion data personalized to the second user in a manner of converting the reference motion data based on information of the second user, and
control the actuator based on the target motion data.
